(19) United States
(12) Patent Application Publication
(10) Pub. No.: US 2023/0181006 A1
(43) Pub. Date: Jun. 15, 2023

(54) IMAGE PICKUP UNIT AND ENDOSCOPE

(71) Applicant: OLYMPUS MEDICAL SYSTEMS CORP., Tokyo (JP)

(72) Inventor: Shinya ISHIKAWA, Tokyo (JP)

(73) Assignee: OLYMPUS MEDICAL SYSTEMS CORP., Tokyo (JP)

(21) Appl. No.: 18/108,310

(22) Filed: Feb. 10, 2023

Related U.S. Application Data
(63) Continuation of application No. PCT/JP2020/043788, filed on Nov. 25, 2020.

(51) Int. Cl. A61B 1/00; A61B 1/05

(52) U.S. Cl. CPC A61B 1/00096; A61B 1/00114; A61B 1/05

(57) ABSTRACT

The image pickup unit includes an image pickup device, a substrate, a movable lens drive member, a flexible substrate and a movable lens drive cable, and an image pickup cable. The flexible substrate, the movable lens drive cable, and the image pickup cable are disposed on the substrate.

### CROSS REFERENCE TO RELATED APPLICATION

This application is a continuation application of PCT/JP2020/043788 filed on Nov. 25, 2020, the entire contents of which are incorporated herein by this reference.

### BACKGROUND OF THE INVENTION

### 1. Field of the Invention

The present invention relates to an image pickup unit and an endoscope including a substrate portion provided to an image pickup device on a side of a back surface of a light receiving surface and electrically connected to the image pickup device, and a movable lens drive portion provided on a side of the light receiving surface of the image pickup device.

### 2. Description of the Related Art

Endoscopes have been widely used in a medical field and industrial field in recent years. By inserting an elongated insertion portion into a subject, an endoscope can pick up an image of an inside of the subject using an image pickup unit provided in the insertion portion.

The image pickup unit includes an objective lens unit. In the objective lens unit, one or multiple optical systems are held by a frame body.

The image pickup unit includes an image pickup device such as CCD or CMOS to pick up an image of a subject that is incident on a light receiving surface via the optical system, on a proximal end side relative to the objective lens unit in an optical axis direction of the optical system.

The image pickup unit includes a substrate portion being electrically connected to the image pickup device and on which an electronic component is mounted, on a side of a back surface of the light receiving surface of the image pickup device, that is, on the proximal end side relative to the image pickup device in the optical axis direction.

The image pickup unit includes an image pickup cable electrically connected to the substrate portion, through which an electric signal is transmitted and received to and from the substrate portion and the image pickup cable supplies electric power to the image pickup device.

There is a well-known configuration of an image pickup unit in which a movable lens drive portion is provided on a side of the light receiving surface of the image pickup device in the optical axis direction.

Specifically, a main portion of the movable lens drive portion is configured to include an objective lens unit and an actuator.

The movable lens drive portion has a configuration in which an optical system in a frame of the objective lens unit is moved back and forth in the optical axis direction by the actuator (hereinafter, an optical system that moves in the optical axis direction is referred to as a movable lens) to vary a focal position of a subject that is incident on the light receiving surface of the image pickup device.

There is a well-known configuration in which a voice coil motor, for example, is used to move a movable lens using an actuator.

As an example, the voice coil motor includes multiple magnets that are provided on an outer periphery of a movable frame being a movable portion to hold a movable lens. The multiple magnets are magnetically polarized at equal intervals in an outer peripheral direction of the movable frame, and such that magnetic poles in a radial direction of the movable frame are the same. The voice coil motor has a configuration in which an electromagnetic coil is wound around an outer periphery of a lens frame holding the movable frame at a position facing the magnet in a radial direction of the lens frame.

With the configuration above, when a current is supplied to the electromagnetic coil, the current acts on a magnetic field generated by the magnet, and the movable frame, that is, the movable lens moves back and forth in the optical axis direction according to Fleming's left-hand rule.

There is also a well-known configuration of a position detection mechanism in which a position detection portion being a Hall effect device or the like facing a magnet provided on an outer periphery of the movable frame in a radial direction is provided inside or outside the lens frame. The position detection portion detects a magnetic field of the facing magnet and detects a position of the movable frame inside the lens frame in the optical axis direction.

As described above, international Publication No. 2014/203626 discloses an image pickup unit having a configuration in which a movable frame is moved back and forth in an optical axis direction using a voice coil motor and a position of the movable frame in the optical axis direction is detected by a position detection portion.

In a case that an image pickup unit including a movable lens drive portion and a position detection portion is provided in an insertion portion of an endoscope, making the insertion portion of the endoscope decrease in diameter demands the image pickup unit to be decreased in diameter.

### SUMMARY OF THE INVENTION

An image pickup unit according to an aspect of the present invention includes an image pickup device, a substrate provided to the image pickup device on a side of a back surface of a light receiving surface and electrically connected to the image pickup device, a movable lens drive member provided to the image pickup device on a side of the light receiving surface, a flexible substrate and a movable lens drive cable to supply electric power to at least the movable lens drive member, and an image pickup cable to supply electric power to the image pickup device. The flexible substrate, the movable lens drive cable, and the image pickup cable are disposed on the substrate.

Further, an endoscope according to an aspect of the present invention includes the image pickup unit.

### BRIEF DESCRIPTION OF THE DRAWINGS

FIG. 1 is an external view of an endoscope including an image pickup unit of a first embodiment.

FIG. 2 is a perspective view of the image pickup unit provided in a distal end portion of an insertion portion of the endoscope in FIG. 1.

FIG. 3 is a sectional view of the image pickup unit taken along a line in FIG. 2.

FIG. 4 is a side view of a member of the image pickup unit in FIG. 2 surrounded by a line IV, viewed from a IV direction in FIG. 2.

FIG. 5 is an enlarged perspective view of a member surrounded by the line IV in FIG. 2 with a flexible substrate removed therefrom.

FIG. 6 is a perspective view of a member in FIG. 5 viewed in a VI direction in FIG. 5.

FIG. 7 is a partial perspective view illustrating a state in which a movable lens drive cable and a temperature compensation circuit output signal cable are electrically connected to a first land in FIG. 2 and an image pickup cable is electrically connected to a second land.

FIG. 8 is a schematic view focusing on a wiring in a substrate, in a partial section of the member in FIG. 5 taken along a line VIII-VIII in FIG. 5.

FIG. 9 is a side view of an image pickup unit according to a second embodiment.

FIG. 10 is an enlarged perspective view of a member surrounded by a line X in FIG. 9.

FIG. 11 is a perspective view of the member in FIG. 10 viewed in an XI direction in FIG. 10.

FIG. 12 is a partial perspective view illustrating a state in which a movable lens drive cable and a temperature compensation circuit output signal cable are electrically connected to a first land in FIG. 10 and an image pickup cable is electrically connected to a second land in FIG. 11.

FIG. 13 is a schematic view focusing on a wiring in a substrate in a partial section of the member in FIG. 10 taken along a line XIII-XIII in FIG. 10.

FIG. 14 is a side view of an image pickup unit according to a third embodiment.

FIG. 15 is an enlarged perspective view of a member surrounded by a line XV in FIG. 14.

FIG. 16 is a perspective view of the member in FIG. 15 viewed in a XVI direction in FIG. 15.

FIG. 17 is a partial perspective view illustrating a state in which a movable lens drive cable and a temperature compensation circuit output signal cable are electrically connected to a first land in FIG. 14 and an image pickup cable is electrically connected to a second land in FIG. 16.

FIG. 18 is a schematic view illustrating a wiring in a substrate, a flexible substrate, a drive cable, and an image pickup cable together, focusing on the wiring in the substrate, in a partial section of the member in FIG. 15 taken along a line XVIII-XVIII in FIG. 15.

### DETAILED DESCRIPTION OF THE PREFERRED EMBODIMENT(S)

Hereinafter, embodiments of the present invention will be described with reference to the drawings. It should be noted that the drawings are schematic, and the relationship between the thickness and the width of each member, the ratios of the thicknesses of respective members, and the like are different from actual ones, and it is needless to say that the drawings include portions having dimensional relationships and ratios mutually different from each other.

### FIRST EMBODIMENT

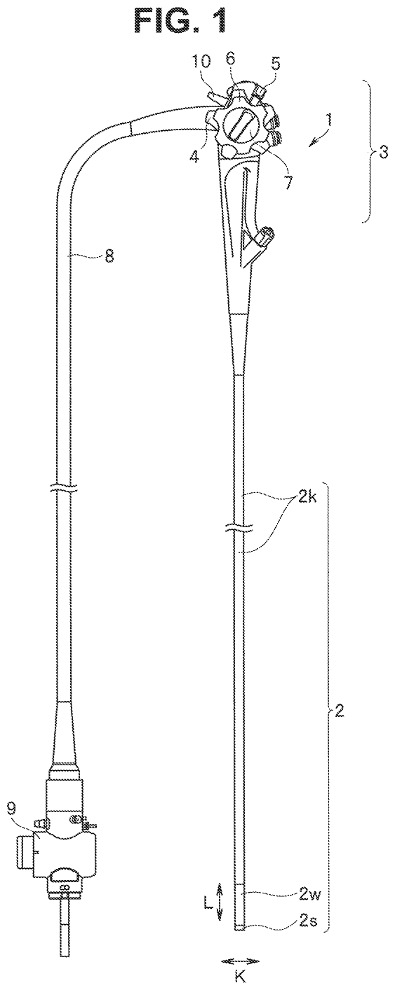

FIG. 1 is an external view of an endoscope including an image pickup unit according to the present embodiment.

As illustrated in FIG. 1, a main portion of an endoscope 1 is configured to include an insertion portion 2 to be inserted into a subject, an operation portion 3 connected to a proximal end side of the insertion portion 2, a universal cord 8 extending from the operation portion 3, and a connector 9 provided at an extending end of the universal cord 8.

Note that the endoscope 1 is electrically connected to an external device such as a control device or an illumination device via the connector 9.

The operation portion 3 is provided with an up-and-down bending operation knob 4 used to bend a bending portion 2w (described later) of the insertion portion 2 in an up-and-down direction, and a left-and-right bending operation knob 6 used to bend the bending portion 2w in a left-and-right direction.

The operation portion 3 is provided with a fixing lever 5 to fix a rotational position of the up-and-down bending operation knob 4 and a fixing knob 7 to fix a rotational position of the left-and-right bending operation knob 6.

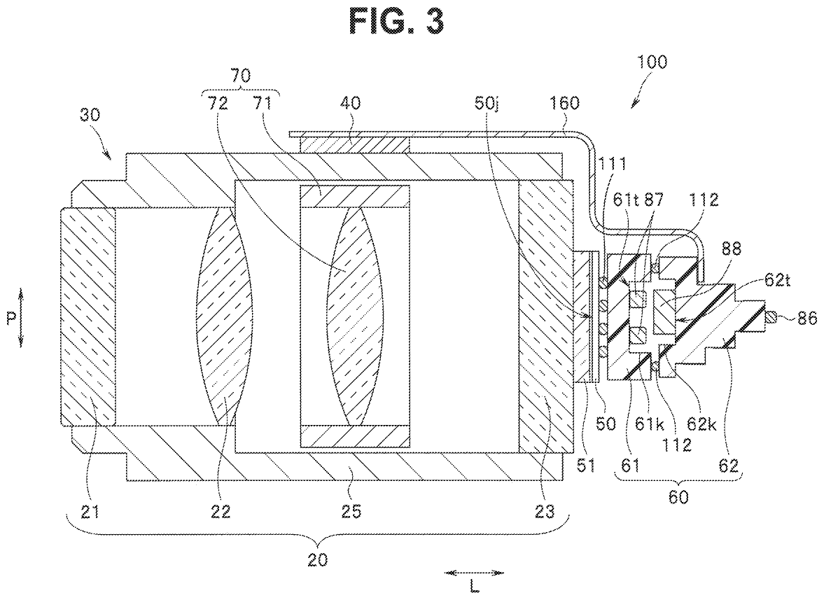

Further, the operation portion 3 is provided with a zoom lever 10 to move a movable frame 71 of an actuator 70 in a movable lens drive portion 30 of an image pickup unit 100 described later (see FIG. 3 for all of the elements except the zoom lever 10).

The insertion portion 2 is configured of a distal end portion 2s, the bending portion 2w, and a flexible tube portion 2k in this order from a distal end side, and has an elongated form.

The bending portion 2w is bent in four directions of up, down, left, and right, for example, by a rotating operation of the up-and-down bending operation knob 4 and the left-and-right bending operation knob 6. With the rotating operation, the bending portion 2w changes an observation direction of the image pickup unit 100 (described later) provided in the distal end portion 2s, or improves insertion easiness of the distal end portion 2s into a subject. Further, the flexible tube portion 2k is connected to a proximal end side of the bending portion 2w.

The image pickup unit 100 is provided in the distal end portion 2s connected to the distal end side of the bending portion 2w.

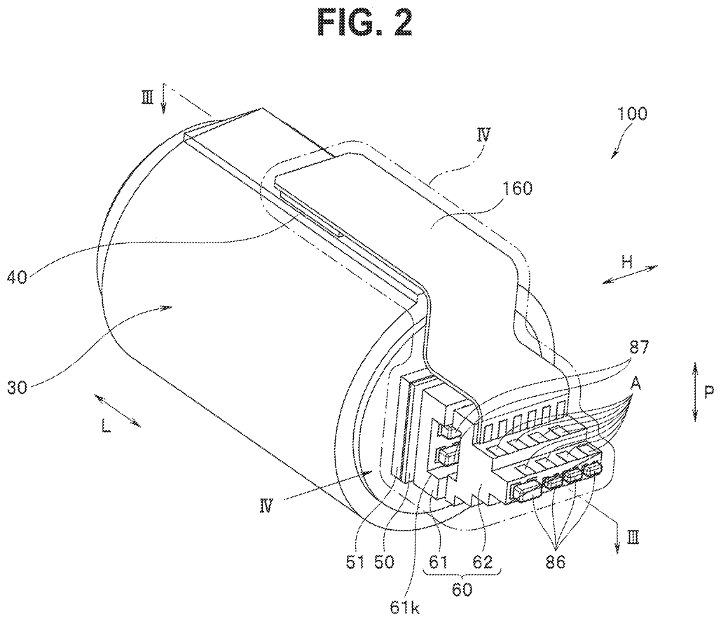

Next, a configuration of the image pickup unit 100 will be described with reference to FIG. 2 to FIG. 8.

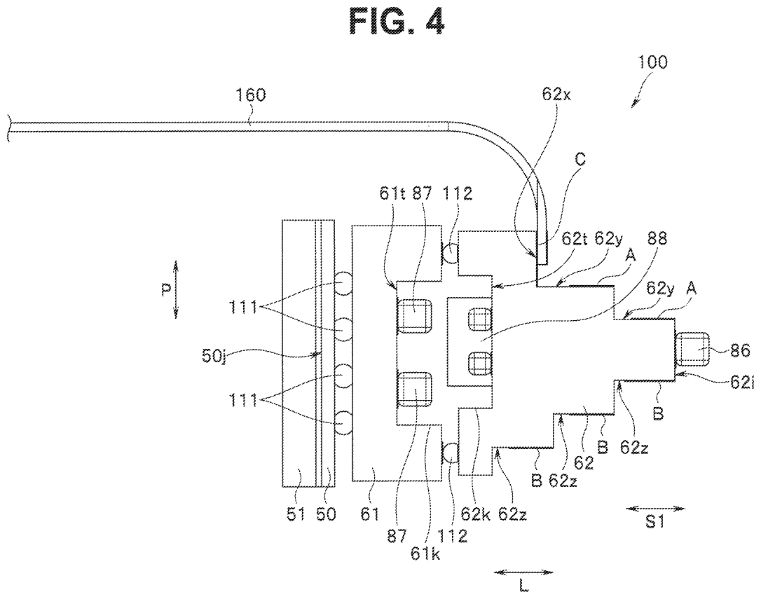

FIG. 2 is a perspective view of the image pickup unit provided in the distal end portion of the insertion portion of the endoscope in FIG. 1, FIG. 3 is a sectional view of the image pickup unit taken along a line in FIG. 2, and FIG. 4 is a side view of a member in the image pickup unit in FIG. 2 surrounded by a line IV, viewed in a IV direction in FIG. 2.

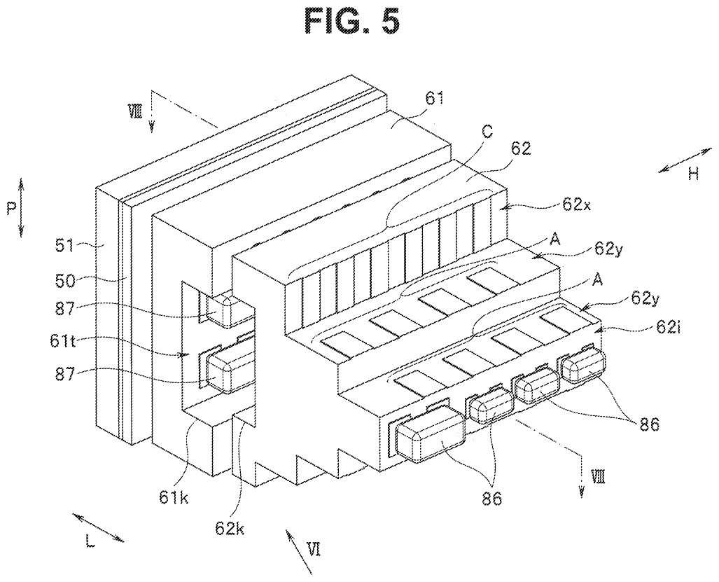

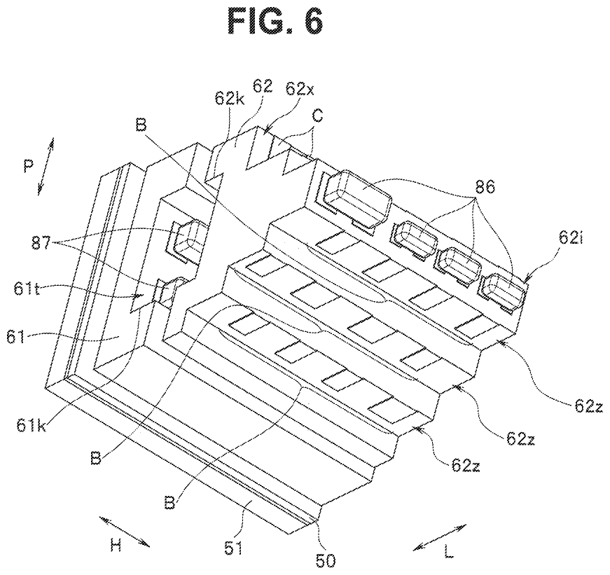

FIG. 5 is an enlarged perspective view of a member surrounded by the line IV in FIG. 2 with a flexible substrate removed therefrom, and FIG. 6 is a perspective view of the member in FIG. 5 viewed in a VI direction in FIG. 5.

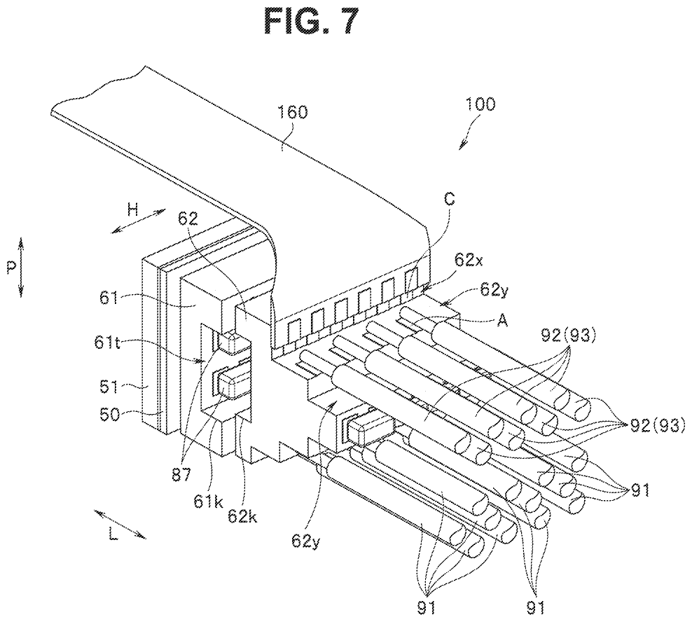

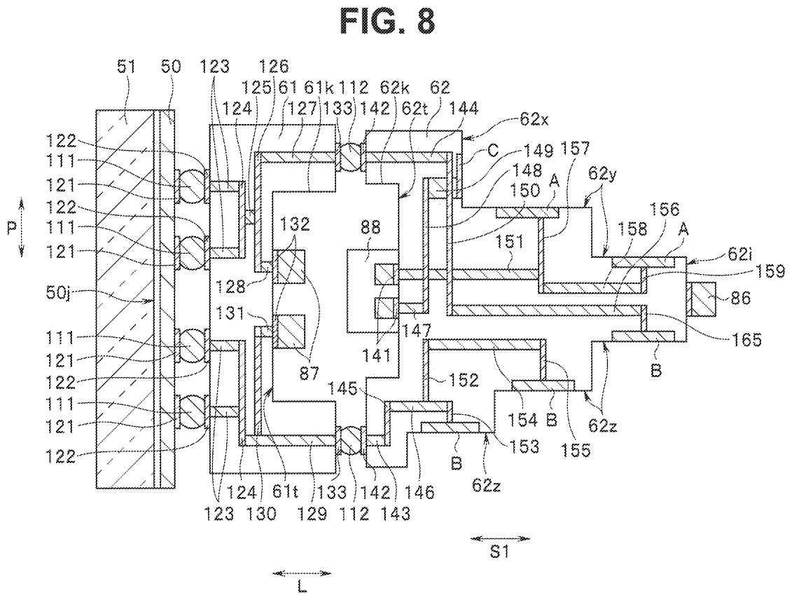

FIG. 7 is a partial perspective view illustrating a state in which a movable lens drive cable and a temperature compensation circuit output signal cable are electrically connected to a first land in FIG. 2 and an image pickup cable is electrically connected to a second land. FIG. 8 is a schematic view focusing on a wiring in a substrate in a partial section of the member in FIG. 5 taken along a line VIII-VIII in FIG. 5.

As illustrated in FIG. 2 to FIG. 8, the image pickup unit 100 includes the movable lens drive portion 30, an image pickup device 50, a substrate portion 60, an electronic component 86, an image pickup electronic component 87, a temperature compensation circuit component 88, an image pickup cable 91, a movable lens drive cable 92, a temperature compensation circuit output signal cable 93, and a flexible substrate 160.

The image pickup device 50 is constituted of CCD, CMOS, or the like, and as illustrated in FIG. 3, picks up an image of a subject that is incident on a light receiving surface 50j via optical systems 21, 22, and 23 described later and a movable lens 72 in the movable lens drive portion 30.

Note that a cover glass 51 to protect the light receiving surface 50j is adhered to the light receiving surface 50j.

The movable lens drive portion 30, which is a movable lens drive member, is provided to the image pickup device 50 on a side of the light receiving surface 50j as illustrated in FIG. 2 and FIG. 3, and a main portion thereof is configured to include an objective lens unit 20 and the actuator 70.

A main portion of the objective lens unit 20 is configured to include the multiple optical systems 21, 22, and 23, and a lens frame 25 to hold the optical systems 21, 22, and 23.

Note that the number of the optical systems held by the lens frame 25 is not limited to three. Further, the objective lens unit 20 may have multiple lens frames. Hereinafter, a direction in which the optical systems 21, 22, and 23 are arranged side by side is referred to as an optical axis direction L.

A surface of the optical system 23 on a proximal end side in the optical axis direction L is adhered to a surface of the cover glass 51 on a distal end side in the optical axis direction L. With the position of the optical system 23, the objective lens unit 20 is provided to the image pickup device 50 on the side of the light receiving surface 50j, that is, on the distal end side in the optical axis direction L.

The actuator 70 is configured of a voice coil motor, for example, and includes a movable lens 72 and a movable frame 71 that is a movable portion to hold the movable lens 72.

Note that, in the lens frame 25, the movable frame 71 and the movable lens 72 are able to move back and forth freely in the optical axis direction L between the optical system 22 and the optical system 23 in the optical axis direction L.

The movable frame 71 is made of a magnetic material having high magnetic permeability, such as SUS-based material or ferrite.

Although not illustrated in order to simplify the drawings, on an outer periphery of the movable frame 71, two magnets, for example, constituting the actuator 70 are provided in an outer peripheral direction of the outer periphery with an interval of 180°. Note that each magnet is magnetically polarized such that the magnetic poles in a radial direction of the movable frame 71 are the same.

Specifically, in each magnet, an N pole is magnetized on an inner side in the radial direction of the movable frame 71, and an S pole is magnetized on an outer side in the radial direction of the movable frame 71, for example.

Note that, as long as the magnets are magnetically polarized such that the magnetic poles in the radial direction of the movable frame 71 are the same, in each magnet, contrary to the above, the S pole may be magnetized on the inner side in the radial direction of the movable frame 71 and the N pole may be magnetized on the outer side in the radial direction of the movable frame 71.

Although not illustrated in order to simplify the drawings, a driving coil configuring the actuator 70 is circumferentially wound around the outer periphery of the lens frame 25 in the optical axis direction L. The length of the driving coil is at least to face each magnet in the radial direction of the movable frame 71. Upon energization, the actuator 70 makes the movable frame 71 move back and forth in the optical axis direction L by acting on the magnetic field of each magnet of the movable frame 71 described above.

With the configuration of the driving coil, when a driving current is supplied to the driving coil, a magnetic field is generated for each magnet, and each magnet acts on the magnetic field to generate a driving force to the movable frame 71 according to Fleming's left-hand rule. Note that, at the energization, only the driving current is supplied to the driving coil.

Then, switching a direction of the current flowing through the driving coil makes the movable frame 71 move back and forth in the lens frame 25 in the optical axis direction L. Accompanying the movement of the movable frame 71, a focal position for a subject in the endoscope 1 is varied.

Note that, since the configuration for moving the movable frame 71 in the optical axis direction L using the driving coil and the magnets other than the above description is well known, a detailed description thereof will be omitted.

Here, as illustrated in FIG. 3, a magnetic sensor 40 is provided on the outer periphery of the lens frame 25 along a moving range of the movable frame 71 in the optical axis direction L, at a position facing the movable frame 71 in the radial direction. The magnetic sensor 40 is a position detection portion to detect a position of the movable frame 71 in the optical axis direction L. Note that an example of the magnetic sensor 40 is a Hall effect device.

The magnetic sensor 40, supplied with not a driving current but only an alternating current, detects the position of the movable frame 71 when the movable frame 71 moves in the optical axis direction L, by detecting an alternating voltage excited by the driving coil, or by detecting an inductance change, for example.

Note that, since the detailed configuration to detect a position by supplying an alternating current is the same as that in a case of using a magnetic sensor such as a Hall effect device in the past, a detailed description thereof will be omitted.

As illustrated in FIG. 2 to FIG. 8, the substrate portion 60, which is a substrate, is provided on a side of a back surface of the light receiving surface 50j of the image pickup device 50, that is, on the proximal end side in the optical axis direction L, and is electrically connected to the image pickup device 50 with a bump 111 made of a solder ball or a gold stud, for example.

In the present embodiment, the substrate portion 60 is configured of a front side substrate portion 61 and a rear side substrate portion 62, and the front side substrate portion 61 and the rear side substrate portion 62 are electrically connected to each other by a bump 112 made of a solder ball or gold stud, for example, in the optical axis direction L.

Note that although not illustrated in detail, each of the front side substrate portion 61 and the rear side substrate portion 62 is formed, for example, by laminating multiple ceramic substrates in a substrate lamination direction S1 illustrated in FIG. 4 and FIG. 7 in parallel with the optical axis direction L, and then by firing and curing the ceramic substrates.

The size of an outer shape of the front side substrate portion 61 is formed substantially the same as or smaller than the size of an outer shape of the image pickup device 50.

As illustrated in FIG. 2 to FIG. 8, a recess 61k is formed to the front side substrate portion 61 on a proximal end side surface facing the rear side substrate portion 62 and being in parallel with the light receiving surface 50j. The recess 61k penetrates through the front side substrate portion 61 in a direction H orthogonal to the optical axis direction L, and has a predetermined depth in the optical axis direction L.

The multiple image pickup electronic components 87, electrically connected to the image pickup device 50, are provided on a bottom surface 61t of the recess 61k in parallel with the light receiving surface 50j, to be accommodated in the recess 61k in the direction H. With the recess 61k, it is prevented that the image pickup electronic component 87 protrudes to the outside of the substrate portion 60 in the radial direction of the substrate portion 60.

The size of an outer shape of the rear side substrate portion 62 is formed substantially the same as or smaller than the size of the outer shape of the front side substrate portion 61. Further, as illustrated in FIG. 2 to FIG. 8, a recess 62k is formed to the rear side substrate portion 62 on a distal end side surface facing the front side substrate portion 61 and being in parallel with the light receiving surface 50j. The recess 62k penetrates through the rear side substrate portion 62 in the direction H, and has a predetermined depth in the optical axis direction L.

The temperature compensation circuit component 88 is provided on a bottom surface 62t of the recess 62k in parallel with the light receiving surface 50j. In other words, the temperature compensation circuit component 88 is accommodated in the recess 62k. With the recess 62k, it is prevented that the temperature compensation circuit component 88 protrudes to the outside of the substrate portion 60 in the radial direction of the substrate portion 60.

The temperature compensation circuit component 88 is electrically connected to the magnetic sensor 40 via the flexible substrate 160, and performs temperature compensation of an output signal of the magnetic sensor 40. Note that, since the function of the temperature compensation circuit component 88 is well known, a detailed description thereof will be omitted.

As illustrated in FIG. 2 to FIG. 8, an outer surface of the rear side substrate portion 62 is formed in a stepped shape, for example, a three stepped shape such that the size of the outer shape decreases toward the proximal end side in the optical axis direction L. Note that the electronic component 86 is provided on a surface 62i of the rear side substrate portion 62 on the proximal end side in the optical axis direction L.

Here, as illustrated in FIG. 5, on a stepped shape outer surface of the rear side substrate portion 62, multiple first lands A are provided in the direction H with a predetermined pitch, on a first surface 62y that is a surface perpendicular to the light receiving surface 50j.

As illustrated in FIG. 7, respective tip ends of the multiple movable lens drive cables (hereinafter simply referred to as drive cables) 92 and temperature compensation circuit output signal cables (hereinafter simply referred to as signal cables) 93 are electrically connected to the multiple first lands A.

Note that, although eight first lands A are illustrated and eight drive cables 92 and signal cables 93 are illustrated in FIG. 7, the number of the first lands A is not limited to eight, and the number of the drive cables 92 and signal cables 93 is not limited to eight.

The eight drive cables 92 and signal cables 93 are constituted of two drive cables and six signal cables, for example.

The drive cables 92 are inserted into the insertion portion 2, the operation portion 3, the universal cord 8, and the connector 9, and supply electric power to the movable lens drive portion 30.

The signal cables 93 are inserted into the insertion portion 2, the operation portion 3, the universal cord 8, and the connector 9, and transfer an output signal of the temperature compensation circuit component 88.

Note that since the drive cables 92 and the signal cables 93 are electrically connected to the first lands A formed on a stepped shape outer surface of the substrate portion 60, the drive cables 92, the signal cables 93, and the first lands A overlap with the image pickup device 50 in a plan view of the image pickup device 50 in the optical axis direction L. Thus, the image pickup unit 100 has a configuration in which an increase in diameter is prevented.

As illustrated in FIG. 6, on the stepped shape outer surface of the rear side substrate portion 62, multiple second lands B are provided in the direction H with a predetermined pitch on a second surface 62z. The second surface 62z is a surface perpendicular to the light receiving surface 50j, on a side opposite to the first surface 62y in a direction P orthogonal to the optical axis direction L and the direction H.

Further, as illustrated in FIG. 7, respective tip ends of the multiple image pickup cables 91 are electrically connected to the multiple second lands B.

Note that although twelve second lands B and twelve image pickup cables 91 are illustrated in FIG. 7, the number of the second lands B is not limited to twelve and the number of the image pickup cables 91 is not limited to twelve.

The image pickup cables 91 are inserted into the insertion portion 2, the operation portion 3, the universal cord 8, and the connector 9, and supply electric power to the image pickup device 50.

Note that, since the image pickup cables 91 are electrically connected to the second lands B formed on the stepped shape outer surface of the substrate portion 60, the image pickup cables 91 and the second lands B overlap with the image pickup device 50 in a plan view of the image pickup device 50 in the optical axis direction L. Thus, the image pickup unit 100 has a configuration in which an increase in diameter is prevented.

The first land A and the second land B are exposed on the outer surface of the rear side substrate portion 62 in a direction orthogonal to the substrate lamination direction S1 that is a formation direction of a via to be described later, and serve as lands to which respective cables are electrically connected. Methods of forming the first land A and the second land B include a known half-split via to divide a via into halves in the optical axis direction L, castellation, or the like.

As illustrated in FIG. 5, multiple third lands C are provided in the direction H with a predetermined pitch on a surface 62x in parallel with the light receiving surface 50j, on a side of the first surface 62y of the stepped shape outer surface of the rear side substrate portion 62. Note that the third lands C are provided at positions overlapping with the image pickup device 50 in a state of a plan view of the image pickup device 50 in the optical axis direction L.

The third lands C are provided closer than the first lands A and the second lands B to the image pickup device 50 in the optical axis direction L. With the position of the third lands C, since a distance between the third lands C and the magnetic sensor 40 in the optical axis direction L may be set short, the length of the flexible substrate 160 in the optical axis direction L may be set short.

As illustrated in FIG. 2 to FIG. 4 and FIG. 7, respective terminals of the flexible substrate 160 on a proximal end side are electrically connected to the multiple third lands C. Note that, as illustrated in FIG. 3, terminals of the flexible substrate 160 on the distal end side are electrically connected to the magnetic sensor 40.

The flexible substrate 160 supplies electric power to the movable lens drive portion 30, and in addition, transfers an output signal of the magnetic sensor 40.

Note that the magnetic sensor 40 is electrically connected to the driving coil described above by a cable (not illustrated) or the like. Thus, the flexible substrate 160 supplies electric power (driving current) to the driving coil via the magnetic sensor 40 and the cable.

Note that the third lands C are formed on the surface 62x with a fine pitch (small pitch) by printing or the like. Specifically, the third lands C are formed with a finer pitch than the first lands A and the second lands B formed by the half-split via, the castellation, or the like described above.

The above is because the third lands C may be formed by printing without using the half-split via, the castellation, or the like described above, being formed on the surface 62x perpendicular to the substrate lamination direction S1. Further, with the forming method of such as the first lands A and the second lands B using the half-split via, the pitch between the vias in the direction H cannot be formed to be short.

As described above, in the present embodiment, the flexible substrate 160, the drive cable 92, the signal cable 93, and the image pickup cable 91 are disposed on the rear side substrate portion 62 of the substrate portion 60.

More specifically, the flexible substrate 160, the drive cable 92, the signal cable 93, and the image pickup cable 91 are electrically connected to the rear side substrate portion 62.

Next, with reference to FIG. 8, there will be described a connection configuration of the first land A and the temperature compensation circuit component 88, a connection configuration of the second land B or the image pickup electronic component 87 and the image pickup device 50, and a connection configuration of the temperature compensation circuit component 88 and the third land C.

As illustrated in FIG. 8, first, the connection of the first land A and the temperature compensation circuit component 88 is achieved by electrically connecting a land 141 of the temperature compensation circuit component 88 to the first land A through vias 151 and 158, and connection wirings 157 and 159. The vias 151 and 158 are formed in the substrate lamination direction S1 in the rear side substrate portion 62, and the connection wirings 157 and 159 are formed orthogonal to the substrate lamination direction S1 in the rear side substrate portion 62.

Next, the connection of the second land B and the image pickup device 50 is achieved by electrically connecting lands 121 of the image pickup device 50 and lands 122 of the front side substrate portion 61 on the distal end side in the optical axis direction L, with the bumps 111. The lands 122 and lands 133 of the front side substrate portion 61 on the proximal end side in the optical axis direction L are electrically connected to each other through vias 123, 125, 127, and 129, and connection wirings 124 and 126. The vias 123, 125, 127, and 129 are formed in the substrate lamination direction S1 in the front side substrate portion 61, and the connection wirings 124 and 126 are formed orthogonal to the substrate lamination direction S1 in the front side substrate portion 61. The lands 133 and lands 142 of the rear side substrate portion 62 are electrically connected with the bumps 112. The lands 142 and the second lands B are electrically connected to each other through vias 143, 144, 146, 154, and 156, and connection wirings 145, 150, 152, 153, 155, and 165. The vias 143, 144, 146, 154, and 156 are formed in the substrate lamination direction S1 in the rear side substrate portion 62, and the connection wirings 145, 150, 152, 153, 155, and 165 are formed orthogonal to the substrate lamination direction S1 in the rear side substrate portion 62. As described above, the second lands B and the image pickup device 50 are connected to each other.

The connection of the image pickup electronic component 87 and the image pickup device 50 is achieved by electrically connecting the lands 121 of the image pickup device 50 and the lands 122 of the front side substrate portion 61 with the bumps 111, and electrically connecting the lands 122 and lands 132 of the image pickup electronic component 87 through the vias 123 and 125, vias 128 and 131, and the connection wirings 124 and 126, a connection wiring 130. The vias 123, 125, 128, and 131 are formed in the substrate lamination direction S1 in the front side substrate portion 61, and the connection wirings 124, 126, and 130 are formed orthogonal to the substrate lamination direction S1 in the front side substrate portion 61.

Finally, the connection of the temperature compensation circuit component 88 and the third land C is achieved by electrically connecting the land 141 of the temperature compensation circuit component 88 to the third land C through vias 147 and 149, and a connection wiring 148. The vias 147 and 149 are formed in the substrate lamination direction S1 in the rear side substrate portion 62, and the connection wiring 148 is formed perpendicular to the substrate lamination direction S1 in the rear side substrate portion 62.

With the connection configuration described above, the drive cable 92 and the signal cable 93, electrically connected to the first land A, are electrically connected to the temperature compensation circuit component 88, the third land C, and the flexible substrate 160 electrically connected to the third land C.

The image pickup cable 91, electrically connected to the second land B, is electrically connected to the image pickup device 50.

The flexible substrate 160, electrically connected to the third land C, is electrically connected to the temperature compensation circuit component 88, the first land A, and the drive cable 92 and the signal cable 93 that are electrically connected to the first land A.

Note that other configurations of the image pickup unit 100 is the same as those of the image pickup unit in the past.

As described above, in the present embodiment, the drive cable 92 and the signal cable 93 are electrically connected to the first land A of the rear side substrate portion 62 of the substrate portion 60, the image pickup cable 91 is electrically connected to the second land B, and the flexible substrate 160 is electrically connected to the third land C.

With the connection configuration above, all of the first land A to which the drive cable 92 and the signal cable 93 are electrically connected, the second land B to which the image pickup cable 91 is electrically connected, and the third land C to which the flexible substrate 160 is electrically connected are provided on the substrate portion 60. Further, the first land A, the second land B, and the third land C are disposed at positions overlapping with the image pickup device 50 in a plan view of the image pickup device 50 in the optical axis direction L.

For the reason above, unlike the related art, the drive cable 92 and the signal cable 93 connected to the first land A and the flexible substrate 160 connected to the third land C are not positioned to greatly protrude to the outside of the movable lens drive portion 30 in the radial direction. Thus, a decrease of the image pickup unit 100 in diameter may be achieved.

As described above, with the configuration in which electric power is supplied from the drive cable 92 to the movable lens drive portion 30 to make the movable lens 72 move, the image pickup unit 100 and the endoscope 1 that are decreased in diameter may be provided.

### SECOND EMBODIMENT

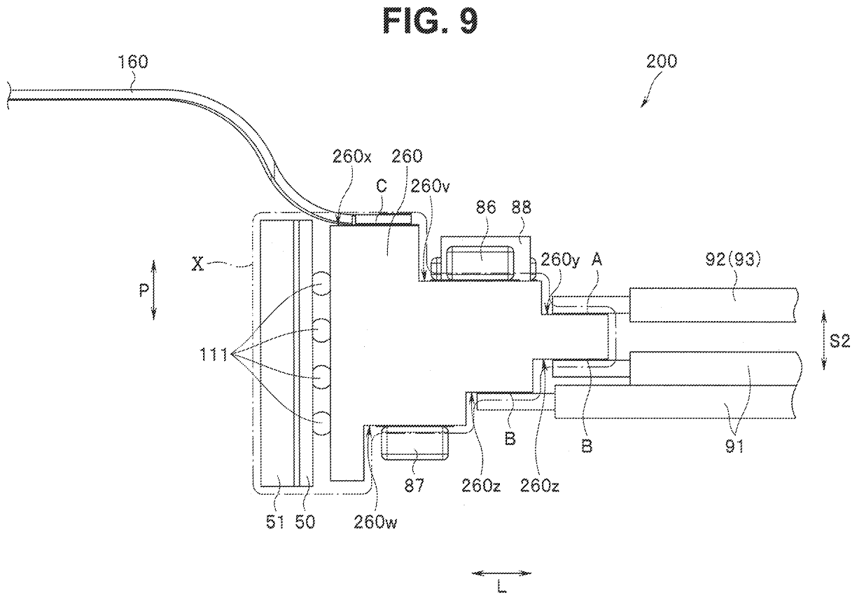

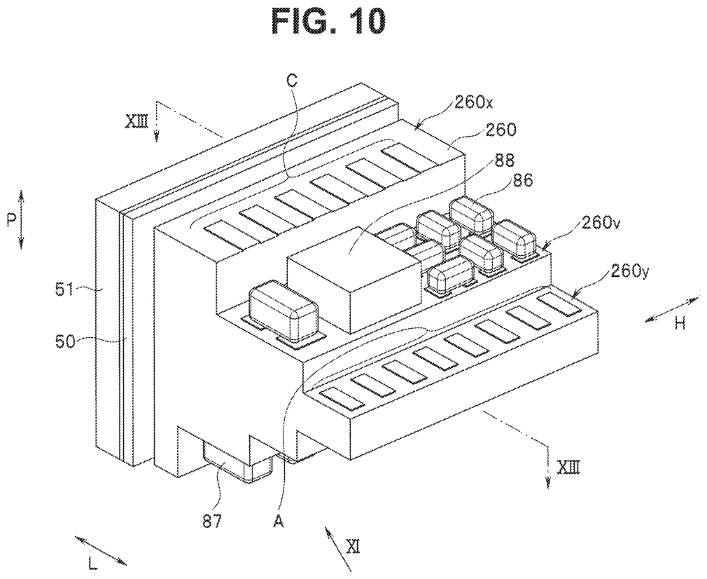

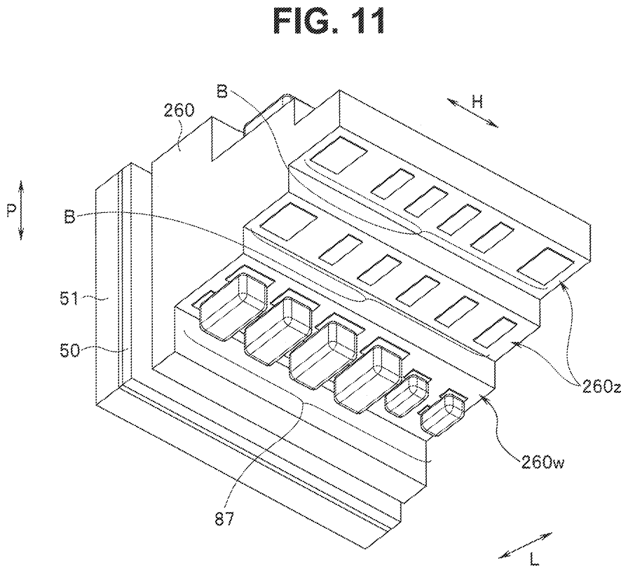

FIG. 9 is a side view of the image pickup unit according to the present embodiment, FIG. 10 is an enlarged perspective view of a member surrounded by a line X in FIG. 9, and FIG. 11 is a perspective view of a member in FIG. 10 viewed in an XI direction in FIG. 10.

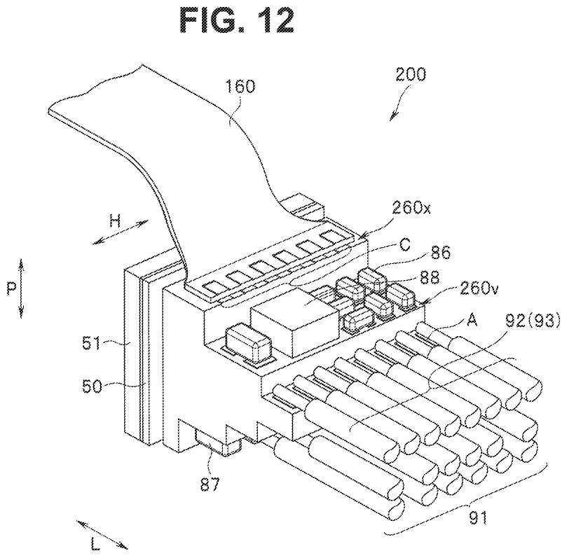

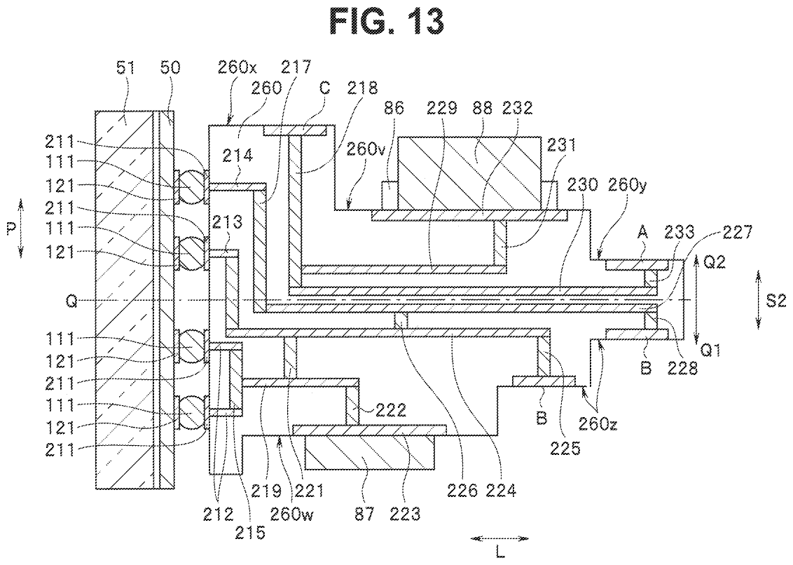

FIG. 12 is a partial perspective view illustrating a state in which a movable lens drive cable and a temperature compensation circuit output signal cable are electrically connected to a first land in FIG. 10 and an image pickup cable is electrically connected to a second land in FIG. 11. FIG. 13 is a schematic view focusing on a wiring in a substrate, in a partial section of the member in FIG. 10 taken along a line XIII-XIII in FIG. 10.

The configuration of the image pickup unit and the endoscope of the second embodiment is different from that of the image pickup unit and the endoscope of the first embodiment illustrated in FIG. 1 to FIG. 8 described above in that a substrate portion is constituted of one piece, in a formation position of a third land relative to the substrate portion, and in a position on the substrate portion where a temperature compensation circuit component is provided.

Thus, only the difference above will be described, and the same configurations as those of the first embodiment will be denoted by the same reference signs, and a description thereof will be omitted.

As illustrated in FIG. 9 to FIG. 13, an image pickup unit 200 includes a substrate portion 260.

Note that, other than the substrate portion 260, configurations of the movable lens drive portion 30, the image pickup device 50, the electronic component 86, the image pickup electronic component 87, the temperature compensation circuit component 88, the image pickup cable 91, the drive cable 92, the signal cable 93, and the flexible substrate 160 constituting the image pickup unit 200 are the same as those in the first embodiment described above, and thus a description thereof will be omitted.

As illustrated in FIG. 9 to FIG. 13, the substrate portion 260 is provided on the side of the back surface of the light receiving surface 50j of the image pickup device 50, that is, on the proximal end side in the optical axis direction L, and is electrically connected to the image pickup device 50 with the bump 111, for example. Note that, in the present embodiment, the substrate portion 260 is constituted of one member.

Although not illustrated in detail, the substrate portion 260 is formed, for example, by laminating multiple ceramic substrates in a substrate lamination direction S2 illustrated in FIG. 9 and FIG. 13 perpendicular to the optical axis direction L, and then by firing and curing the ceramic substrates.

The size of an outer shape of the substrate portion 260 is formed substantially the same as or smaller than the size of the outer shape of the image pickup device 50.

As illustrated in FIG. 9 to FIG. 13, an outer surface of the substrate portion 260 is formed in a stepped shape, for example, a three stepped shape such that the size of the outer shape decreases toward the proximal end side in the optical axis direction L.

Here, as illustrated in FIG. 9, FIG. 10, FIG. 12, and FIG. 13, on a stepped shape outer surface of the substrate portion 260, multiple first lands A are provided in the direction H with a predetermined pitch, on a first surface 260y that is a surface perpendicular to the light receiving surface 50j.

As illustrated in FIG. 9 and FIG. 12, respective tip ends of the multiple drive cables 92 and signal cables 93 are electrically connected to the multiple first lands A.

Note that although eight first lands A are illustrated and eight drive cables 92 and signal cables 93 are illustrated in FIG. 12, the number of the first lands A is not limited to eight, and the number of the drive cables 92 and signal cables 93 is not limited to eight.

The eight drive cables 92 and signal cables 93 are constituted of two drive cables and six signal cables, for example.

Note that since the drive cables 92 and the signal cables 93 are electrically connected to the first lands A formed on the stepped shape outer surface of the substrate portion 260, the drive cables 92, the signal cables 93, and the first lands A overlap with the image pickup device 50 in a plan view of the image pickup device 50 in the optical axis direction L. Thus, the image pickup unit 200 has a configuration in which an increase in diameter is prevented.

On a side of the first surface 260y of the stepped shape outer surface of the substrate portion 260, the temperature compensation circuit component 88 and another electronic component 86 are provided in the direction H on a surface 260v. The surface 260v is perpendicular to the light receiving surface 50j, and is closer than the first surface 260y to the image pickup device 50 in the optical axis direction L.

Note that since the temperature compensation circuit component 88 and the other electronic component 86 are provided on the surface 260v formed on the stepped shape outer surface of the substrate portion 260, the temperature compensation circuit component 88 and the other electronic component 86 overlap with the image pickup device 50 in a plan view of the image pickup device 50 in the optical axis direction L. Thus, the image pickup unit 200 has a configuration in which an increase in diameter is prevented.

As illustrated in FIG. 9, FIG. 11, and FIG. 13, on the stepped shape outer surface of the substrate portion 260, the multiple second lands B are provided in the direction H with a predetermined pitch on a second surface 260z. The second surface 260z is a surface perpendicular to the light receiving surface 50j on a side opposite to the first surface 260y across a center Q of the substrate portion 260 in the direction P.

As illustrated in FIG. 12, respective tip ends of the multiple image pickup cables 91 are electrically connected to the multiple second lands B.

Note that although twelve second lands B and twelve image pickup cables 91 are illustrated in FIG. 12, the number of the second lands B is not limited to twelve and the number of the image pickup cables 91 is not limited to twelve.

Note that since the image pickup cables 91 are electrically connected to the second lands B formed on the stepped shape outer surface of the substrate portion 260, the image pickup cables 91 and the second lands B overlap with the image pickup device 50 in a plan view of the image pickup device 50 in the optical axis direction L. Thus, the image pickup unit 200 has a configuration in which an increase in diameter is prevented.

On a side of the second surface 260z of the stepped shape outer surface of the substrate portion 260, the multiple image pickup electronic components 87, electrically connected to the image pickup device 50, are provided in the direction H on a surface 260w. The surface 260w is perpendicular to the light receiving surface 50j, and is closer than the second surface 260z to the image pickup device 50 in the optical axis direction L.

Note that since the image pickup electronic components 87 are provided on the surface 260w formed on the stepped shape outer surface of the substrate portion 260, the image pickup electronic components 87 overlap with the image pickup device 50 in a plan view of the image pickup device 50 in the optical axis direction L. Thus, the image pickup unit 200 has a configuration in which an increase in diameter is prevented.

As illustrated in FIG. 9, FIG. 10, FIG. 12, and FIG. 13, on a side of the first surface 260y of the stepped shape outer surface of the substrate portion 260, the multiple third lands C are provided in the direction H with a predetermined pitch on a third surface 260x. The third surface 260x is a surface perpendicular to the light receiving surface 50j, and is closer than the surface 260v to the image pickup device 50 in the optical axis direction L. Note that the third lands C are provided at positions overlapping with the image pickup device 50 in a state of a plan view of the image pickup device 50 in the optical axis direction L.

Also in the present embodiment, the third lands C are provided closer than the first lands A and the second lands B to the image pickup device 50 in the optical axis direction L.

As illustrated in FIG. 9 and FIG. 12, respective terminals of the flexible substrate 160 on the proximal end side are electrically connected to the multiple third lands C.

Note that, in the present embodiment, the first lands A, the second lands B, and the third lands C are respectively formed on the first surface 260y, the second surface 260z, and the third surface 260x with a fine pitch by printing or the like.

The above is because the first lands A, the second lands B, and the third lands C may be formed by printing for a reason described above, being respectively formed on the surfaces 260y, 260z, and 260x perpendicular to the substrate lamination direction S2.

As described above, in the present embodiment, the flexible substrate 160, the drive cable 92, the signal cable 93, and the image pickup cable 91 are arranged on the substrate portion 260.

More specifically, the flexible substrate 160, the drive cable 92, the signal cable 93, and the image pickup cable 91 are electrically connected to the substrate portion 260.

Next, with reference to FIG. 13, there will be described a connection configuration of the first land A and the temperature compensation circuit component 88 or the third land C, a connection configuration of the second land B or the image pickup electronic component 87 and the image pickup device 50, and a connection configuration of the temperature compensation circuit component 88 and the third land C.

As illustrated in FIG. 13, first, the connection of the first land A and the temperature compensation circuit component 88 is achieved by electrically connecting a land 232 of the temperature compensation circuit component 88 to the first land A through vias 218, 231, and 233, and connection wirings 229 and 230. The vias 218, 231, and 233 are formed in the substrate lamination direction S2 in the substrate portion 260, and the connection wirings 229 and 230 are formed orthogonal to the substrate lamination direction S2 in the substrate portion 260.

Next, the connection of the first land A and the third land C is achieved by electrically connecting the first land A to the third land C through the vias 218 and 233 and the connection wiring 230. The vias 218 and 233 are formed in the substrate lamination direction S2 in the substrate portion 260, and the connection wiring 230 is formed orthogonal to the substrate lamination direction S2 in the substrate portion 260.

Next, the connection of the second land B and the image pickup device 50 is achieved by electrically connecting the lands 121 of the image pickup device 50 and lands 211 of the substrate portion 260 on the distal end side in the optical axis direction L with the bumps 111, and by electrically connecting the lands 211 and the second lands B through vias 217, 225, 226, and 228, and connection wirings 213, 214, 224, and 227. The vias 217, 225, 226, and 228 are formed in the substrate lamination direction S2 in the substrate portion 260, and the connection wirings 213, 214, 224, and 227 are formed orthogonal to the substrate lamination direction S2 in the substrate portion 260.

The connection of the image pickup electronic component 87 and the image pickup device 50 is achieved by electrically connecting the lands 121 of the image pickup device 50 and the lands 211 of the substrate portion 260 on the distal end side in the optical axis direction L with the bumps 111, and by electrically connecting the lands 211 and a land 223 of the image pickup electronic component 87 through vias 215, 216, 221, and 222, and connection wirings 212, 213, 219, and 224. The vias 215, 216, 221, and 222 are formed in the substrate lamination direction S2 in the substrate portion 260, and the connection wirings 212, 213, 219, and 224 are formed orthogonal to the substrate lamination direction S2 in the substrate portion 260.

Finally, the connection of the temperature compensation circuit component 88 and the third land C is achieved by electrically connecting the land 232 of the temperature compensation circuit component 88 and the third land C, through the vias 218 and 231 formed in the substrate lamination direction S2 in the substrate portion 260, and the connection wiring 229 formed orthogonal to the substrate lamination direction S2 in the substrate portion 260.

As described above, the drive cable 92 and the signal cable 93, electrically connected to the first land A, are electrically connected to the temperature compensation circuit component 88, the third land C, and the flexible substrate 160 electrically connected to the third land C.

The image pickup cable 91, electrically connected to the second land B, is electrically connected to the image pickup device 50.

The flexible substrate 160, electrically connected to the third land C, is electrically connected to the temperature compensation circuit component 88, the first land A, and the drive cable 92 and the signal cable 93 that are electrically connected to the first land A.

Note that other configurations of the image pickup unit 200 are the same as those of the image pickup unit 100 of the first embodiment described above.

With the configuration above, the same effect as that of the first embodiment described above may be obtained. Further, in the present embodiment, since the substrate portion 260 is constituted of one member, manufacturing costs may be reduced as compared with the first embodiment, and in addition, wirings in the substrate portion 260 may be simplified.

Further, on the outer surface of the substrate portion 260, the second land B is formed on the opposite side of the first land A and the third land C across the center Q of the substrate portion 260 in the direction P. With the positions where the lands are formed, in the substrate portion 260, wirings on the image pickup cable 91 side and wirings on the drive cable 92 and signal cable 93 side may be separately designed, that is, independently designed, and thus the wirings may be simplified as compared with the first embodiment.

Furthermore, since the wirings on the image pickup cable 91 side and the wirings on the drive cable 92 and signal cable 93 side may be separated from each other across the center Q of the substrate portion 260 in the direction P, it is less likely to be affected by electrical noise between the wirings, and each cable may obtain appropriate electrical characteristics.

### THIRD EMBODIMENT

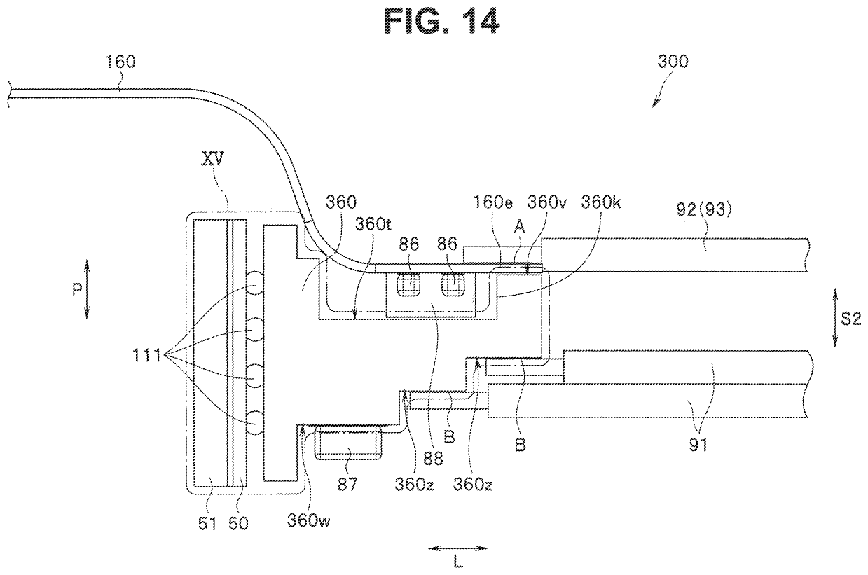

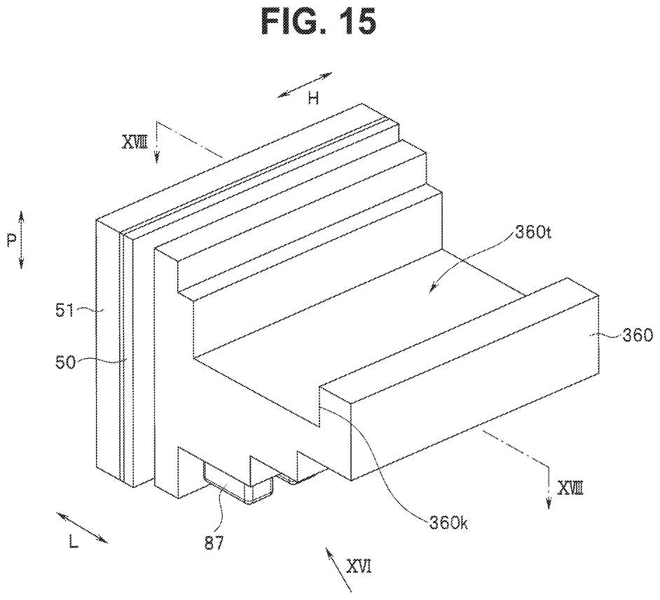

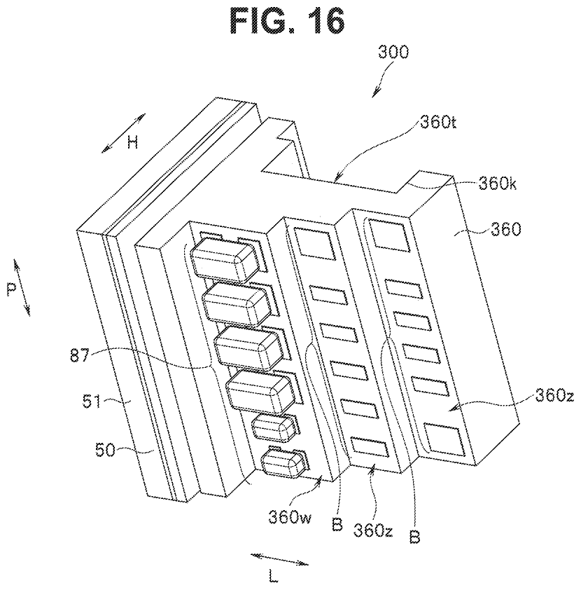

FIG. 14 is a side view of the image pickup unit according to the present embodiment, FIG. 15 is an enlarged perspective view of a member surrounded by a line XV in FIG. 14, and FIG. 16 is a perspective view of the member in FIG. 15 viewed in a XVI direction in FIG. 15.

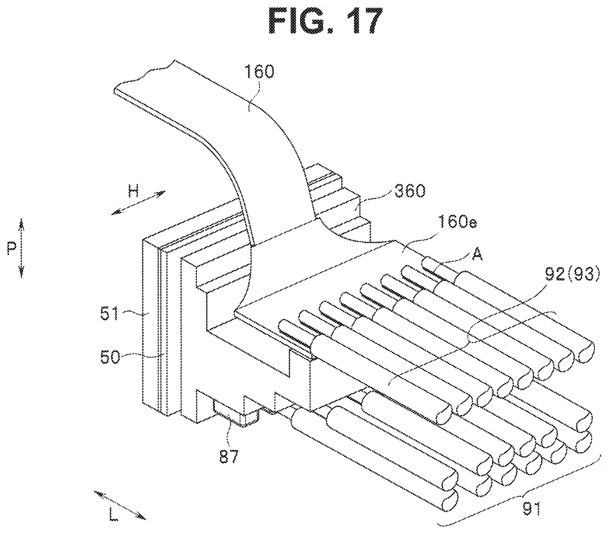

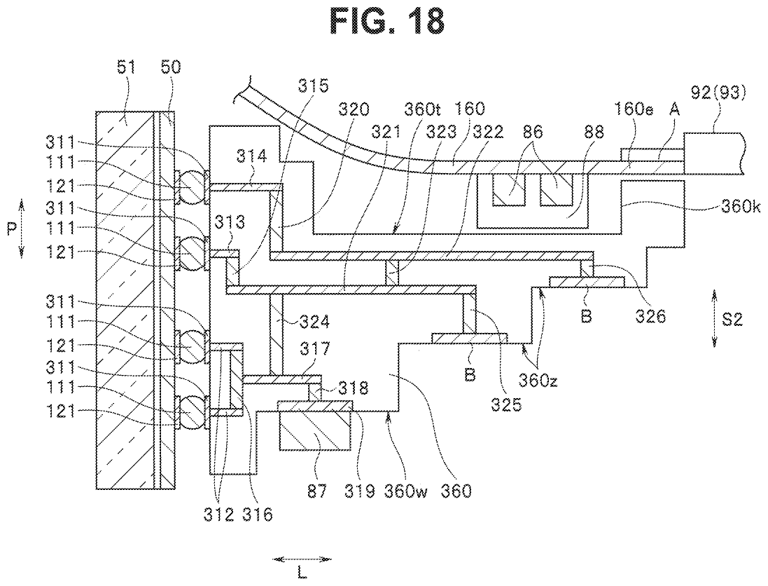

FIG. 17 is a partial perspective view illustrating a state in which a movable lens drive cable and a temperature compensation circuit output signal cable are electrically connected to a first land in FIG. 14, and an image pickup cable is electrically connected to a second land in FIG. 16. FIG. 18 is a schematic view illustrating a wiring in a substrate, a flexible substrate, a drive cable, and an image pickup cable together, focusing on the wiring in the substrate, in a partial section of the member in FIG. 15 taken along a line XVIII-XVIII in FIG. 15.

The configuration of the image pickup unit and the endoscope of the third embodiment is different from that of the image pickup unit and the endoscope of the second embodiment illustrated in FIG. 9 to FIG. 13 described above in that the first land is provided on a flexible substrate, in that a third land is not provided on a substrate portion, and in a position on the substrate portion where a temperature compensation circuit component is provided.

Thus, only the difference above will be described, and the same configurations as those of the second embodiment will be denoted by the same reference signs, and a description thereof will be omitted.

As illustrated in FIG. 14 to FIG. 18, the image pickup unit 300 includes a substrate portion 360.

Note that, other than the substrate portion 360, configurations of the movable lens drive portion 30, the image pickup device 50, the electronic component 86, the image pickup electronic component 87, the temperature compensation circuit component 88, the image pickup cable 91, the drive cable 92, and the flexible substrate 160 constituting the image pickup unit 300 are the same as those in the first and second embodiments described above, and thus a description thereof will be omitted.

As illustrated in FIG. 14 to FIG. 18, the substrate portion 360 is provided on the side of the back surface of the light receiving surface 50j of the image pickup device 50, that is, on the proximal end side in the optical axis direction L, and is electrically connected to the image pickup device 50 with the bump 111, for example. Note that, also in the present embodiment, the substrate portion 360 is constituted of one member.

Although not illustrated in detail, the substrate portion 360 is formed, for example, by laminating multiple ceramic substrates in the substrate lamination direction S2 illustrated in FIG. 14 and FIG. 18 perpendicular to the optical axis direction L, and then by firing and curing the ceramic substrates.

The size of an outer shape of the substrate portion 360 is formed substantially the same as or smaller than the size of the outer shape of the image pickup device 50.

As illustrated in FIG. 14 to FIG. 18, an outer surface of the substrate portion 360 is formed in a stepped shape, for example, a three stepped shape such that the size of the outer shape decreases toward the proximal end side in the optical axis direction L.

Here, as illustrated in FIG. 14 to FIG. 16 and FIG. 18, in a stepped shape outer surface of the substrate portion 360, a recess 360k is formed to penetrate through in the direction H, and to have a predetermined depth in the direction P, on a surface 360v perpendicular to the light receiving surface 50j.

In the recess 360k, the temperature compensation circuit component 88 mounted on a proximal end side portion 160e of the flexible substrate 160, and another electronic component 86 are accommodated in the recess 360k to face a bottom surface 360t of the recess 360k. With the recess 360k, it is prevented that the temperature compensation circuit component 88 protrudes to the outside of the substrate portion 360 in the radial direction of the substrate portion 360.

As illustrated in FIG. 17, in the proximal end side portion 160e of the flexible substrate 160 that is close to or in contact with the surface 360v, the multiple first lands A are provided in the direction H with a predetermined pitch on a surface opposite to a surface on which the temperature compensation circuit component 88 and the other electronic component 86 are mounted.

As illustrated in FIG. 14, FIG. 17, and FIG. 18, respective tip ends of the multiple drive cables 92 and signal cables 93 are electrically connected to the multiple first lands A.

At the electrical connection, since the proximal end side portion 160e of the flexible substrate 160 is close to or in contact with the surface 360v, the drive cable 92 and the signal cable 93 overlap with the image pickup device 50 in a plan view of the image pickup device 50 in the optical axis direction L. Thus, the image pickup unit 300 has a configuration in which an increase in diameter is prevented.

Note that although eight first lands A are illustrated and eight drive cables 92 and signal cables 93 are illustrated in FIG. 17, the number of the first lands A is not limited to eight, and the number of the drive cables 92 and signal cables 93 is not limited to eight.

The eight drive cables 92 and signal cables 93 are constituted of two drive cables and six signal cables, for example.

As a result, the drive cable 92 and the signal cable 93, electrically connected to the first land A, are electrically connected to the flexible substrate 160 and the temperature compensation circuit component 88.

As illustrated in FIG. 14 and FIG. 16 to FIG. 18, on the stepped shape outer surface of the substrate portion 360, the multiple second lands B are provided in the direction H with a predetermined pitch on a second surface 360z. The second surface 360z is a surface perpendicular to the light receiving surface 50j, on a side opposite to the recess 360k in the direction P.

As illustrated in FIG. 14 and FIG. 17, respective tip ends of the multiple image pickup cables 91 are electrically connected to the multiple second lands B.

Note that although twelve second lands B and twelve image pickup cables 91 are illustrated in FIG. 16 and FIG. 17 respectively, the number of the second lands B is not limited to twelve and the number of the image pickup cables 91 is not limited to twelve.

Note that since the image pickup cables 91 are electrically connected to the second lands B formed on the stepped shape outer surface of the substrate portion 360, the image pickup cables 91 and the second lands B overlap with the image pickup device 50 in a plan view of the image pickup device 50 in the optical axis direction L. Thus, the image pickup unit 300 has a configuration in which an increase in diameter is prevented.

On a side of the second surface 360z of the stepped shape outer surface of the substrate portion 360, the multiple image pickup electronic components 87, electrically connected to the image pickup device 50, are provided on a surface 360w. The surface 360w is a surface perpendicular to the light receiving surface 50j, and is closer than the second surface 360z to the image pickup device 50 in the optical axis direction L.

Note that since the image pickup electronic components 87 are provided on the surface 360w formed on the stepped shape outer surface of the substrate portion 360, the image pickup electronic components 87 overlap with the image pickup device 50 in a plan view of the image pickup device 50 in the optical axis direction L. Thus, the image pickup unit 300 has a configuration in which an increase in diameter is prevented.

Note that, in the present embodiment, the second lands B are formed on the second surface 360z with a fine pitch by printing or the like. The above is because the second lands B may be formed by printing as described above, being formed on the surface 360z perpendicular to the substrate lamination direction S2.

As described above, in the present embodiment, the flexible substrate 160, the drive cable 92, the signal cable 93, and the image pickup cable 91 are arranged on the substrate portion 360.

More specifically, the image pickup cable 91 is electrically connected to the substrate portion 360, and the drive cable 92 and the signal cable 93 are electrically connected to the flexible substrate 160 that is close to or in contact with the substrate portion 360.

Next, a connection configuration of the second land B or the image pickup electronic component 87 and the image pickup device 50 will be described with reference to FIG. 18.

As illustrated in FIG. 18, first, the connection of the second land B and the image pickup device 50 is achieved by electrically connecting the lands 121 of the image pickup device 50 and lands 311 of the substrate portion 360 on a distal end side in the optical axis direction L with the bumps 111, and by electrically connecting the lands 311 and the second lands B through vias 315, 320, 323, 325, and 326 and connection wirings 313, 314, 321, and 322. The vias 315, 320, 323, 325, and 326 are formed in the substrate lamination direction S2 in the substrate portion 360, and the connection wirings 313, 314, 321, and 322 are formed orthogonal to the substrate lamination direction S2 in the substrate portion 360.

The connection of the image pickup electronic component 87 and the image pickup device 50 is achieved by electrically connecting the lands 121 of the image pickup device 50 and the lands 311 of the substrate portion 360 on the distal end side in the optical axis direction L with the bumps 111, and by electrically connecting the lands 311 and lands 319 of the image pickup electronic component 87 through the via 315, vias 316, 318, and 324, and connection wirings 312 and 317, the connection wiring 313. The vias 315, 316, 318, and 324 are formed in the substrate lamination direction S2 in the substrate portion 360, and the connection wirings 312, 313, and 317 are formed orthogonal to the substrate lamination direction S2 in the substrate portion 360.

As described above, the image pickup cable 91, electrically connected to the second land B, is electrically connected to the image pickup device 50.

Note that other configurations of the image pickup unit 300 are the same as those of the image pickup unit 100 of the first embodiment and the image pickup unit 200 of the second embodiment described above.

Even with the configuration above, it is possible to obtain the same effects as those of the first and second embodiments described above. Further, since the first land and the third land are not provided in the substrate portion 360, it is sufficient that only the wiring on a side of the image pickup cable 91 is formed in the substrate portion 360. For the reason above, the wiring is simplified, the electrical characteristics of each of the wirings on the side of the image pickup cable 91, and the wirings on a side of the drive cable 92 and the signal cable 93 are improved. In addition, the substrate portion 360 may further be decreased in diameter.

It should be noted that the present invention is not limited to the embodiments described above, and can appropriately be modified within a scope not departing from the gist or idea of the invention that can be read from the claims and the entire description, and an insertion tool and an endoscope with such modifications are also included in the technical scope of the present invention.

What is claimed is:

1. An image pickup unit, comprising:
an image pickup device;
a substrate provided to the image pickup device on a side of a back surface of a light receiving surface and electrically connected to the image pickup device;
a movable lens drive member provided to the image pickup device on a side of the light receiving surface;
a flexible substrate and a movable lens drive cable to supply electric power to at least the movable lens drive member; and
an image pickup cable to supply electric power to the image pickup device, wherein
the flexible substrate, the movable lens drive cable, and the image pickup cable are disposed on the substrate.

2. The image pickup unit according to claim 1, further comprising:
a first land to which the movable lens drive cable is electrically connected; and
a second land provided on the substrate and to which the image pickup cable is electrically connected.

3. The image pickup unit according to claim 2, wherein
the substrate is provided with the first land.

4. The image pickup unit according to claim 3, wherein
the substrate is provided with a third land to which the flexible substrate is electrically connected.

5. The image pickup unit according to claim 2, wherein
the flexible substrate is provided with the first land.

6. The image pickup unit according to claim 2, further comprising:
a magnetic sensor configured to detect a position of a movable frame of an actuator provided to the movable lens drive member;
a temperature compensation circuit provided on the substrate and configured to perform temperature compensation of an output signal of the magnetic sensor; and
a temperature compensation circuit output signal cable to transfer an output signal of the temperature compensation circuit, wherein
the flexible substrate further transfers the output signal of the magnetic sensor, and
the temperature compensation circuit output signal cable and the movable lens drive cable are electrically connected to the first land together.

7. The image pickup unit according to claim 2, wherein
the substrate has a surface perpendicular to the light receiving surface of the image pickup device, and
the second land is provided on the surface perpendicular to the light receiving surface of the image pickup device.

8. The image pickup unit according to claim 6, wherein
the substrate has a surface in parallel with the light receiving surface of the image pickup device and a surface perpendicular to the light receiving surface of the image pickup device, and
the temperature compensation circuit is provided either on the surface in parallel with the light receiving surface of the image pickup device or on the surface perpendicular to the light receiving surface of the image pickup device.

9. The image pickup unit according to claim 4, wherein
the third land is provided closer than the first land and the second land to the image pickup device.

10. The image pickup unit according to claim 4, wherein
the substrate has a surface in parallel with the light receiving surface of the image pickup device, and a first surface and a second surface perpendicular to the light receiving surface of the image pickup device,
the first land is provided on the first surface,
the second land is provided on the second surface, and
the third land is provided on the surface in parallel with the light receiving surface of the image pickup device.

11. The image pickup unit according to claim 4, wherein
the substrate has a first surface, a second surface, and a third surface that are perpendicular to the light receiving surface of the image pickup device,
the first land is provided on the first surface,
the second land is provided on the second surface, and
the third land is provided on the third surface.

12. The image pickup unit according to claim 6, wherein
the temperature compensation circuit is provided in a recess provided either to a surface in parallel with the light receiving surface of the image pickup device of the substrate, or to a surface perpendicular to the light receiving surface of the image pickup device of the substrate.

13. The image pickup unit according to claim 4, wherein
the first land and the third land are positioned on an opposite side of the second land across a center of the substrate in a radial direction of the substrate.

14. An endoscope comprising:
the image pickup unit according to claim 1.
